(19) United States
(12) Patent Application Publication
(10) Pub. No.: US 2016/0129003 A1
(43) Pub. Date: May 12, 2016

(54) Pharmaceutical Combinations

(71) Applicants: Samit HIRAWAT, , East Hanover, NJ (US); Emmanuelle DI TOMASO, Cambridge, MA (US)

(72) Inventors: Samit Hirawat, Chatham, NJ (US); Emmanuelle DI TOMASO, Lexington, MA (US)

(73) Assignee: Novartis AG, Basel (CH)

(21) Appl. No.: 14/897,555

(22) PCT Filed: Jun. 16, 2014
(86) PCT No.: PCT/IB2014/062271

§ 371 Date: Dec. 10, 2015

Related U.S. Application Data
(60) Provisional application No. 61/836,347, filed on Jun. 18, 2013.

(51) Int. Cl. A61K 31/506; A61K 31/4545; A61K 31/4439; A61K 31/4745; A61K 31/5377

(52) U.S. Cl. CPC A61K 31/506; A61K 31/4745; A61K 31/5377; A61K 31/4439; A61K 31/4545

(57) ABSTRACT

A pharmaceutical combination comprising: (a) a phosphatidylinositol-3-kinase inhibitor selected from 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and (b) an anaplastic lymphoma kinase inhibitor, particularly for use in the treatment or prevention of a proliferative disease; uses of such a combination in the treatment or prevention of proliferative diseases; to pharmaceutical compositions of the combination of said therapeutic agents and methods of treating a proliferative disease in a subject comprising administering to said subject a therapeutically effective amount of such a combination.

### FIELD OF THE INVENTION

A pharmaceutical combination comprising: (a) a phosphatidylinositol-3-kinase inhibitor selected from 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and (b) an anaplastic lymphoma kinase inhibitor, particularly for use in the treatment or prevention of a proliferative disease. This invention also relates to the uses of such a combination in the treatment or prevention of proliferative diseases; to pharmaceutical compositions of the combination of said therapeutic agents and methods of treating a proliferative disease in a subject comprising administering to said subject a therapeutically effective amount of such a combination.

### BACKGROUND OF THE INVENTION

Phosphatidylinositol 3-kinases (PI-3 kinase or PI3K) comprise a family of lipid and serine/threonine kinases that catalyze the transfer of phosphate to the D-3′ position of inositol lipids to produce phosphoinositol-3-phosphate (PIP), phosphoinositol-3,4-diphosphate (PIP2) and phosphoinositol-3,4,5-triphosphate (PIP3) that, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). Of the two Class 1 PI3Ks, Class 1A PI3Ks are heterodimers composed of a catalytic p110 subunit (α, β, δ isoforms) constitutively associated with a regulatory subunit that can be p85α, p55α, p50α, p853β or p55γ. The Class 1B sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997)); Katso et al., Annu. Rev. Cell Dev. Biol. 17:615-675 (2001)). Consequently, the resultant phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)).

PI-3 kinase inhibitors are useful therapeutic compounds for the treatment of various conditions in humans. Aberrant regulation of PI3K, which often increases survival through Akt activation, is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110α isoform, PIK3CA, and for Akt are amplified and increased protein expression of their gene products has been demonstrated in several human cancers. Furthermore, mutations and translocation of p85α that serve to up-regulate the p85-p110 complex have been described in a few human cancers. Finally, somatic missense mutations in PIK3CA that activate downstream signaling pathways have been described at significant frequencies in a wide diversity of human cancers (Kang et al., Proc. Natl. Acad. Sci. USA 102:802 (2005); Samuels et al., Science 304:554 (2004); Samuels et al., Cancer Cell 7:561-573(2005)). These observations show that deregulation of phosphoinositol-3 kinase and the upstream and downstream components of this signaling pathway is one of the most common deregulations associated with human cancers and proliferative diseases (Parsons et al., Nature 436:792(2005); Hennessey at el., Nature Rev. Drug Dis. 4:988-1004 (2005)).

Further, anaplastic lymphoma kinase (ALK), a member of the insulin receptor superfamily of receptor tyrosine kinases, has been implicated in oncogenesis in hematopoietic and non-hematopoietic tumors. The aberrant expression of full-length ALK receptor proteins has been reported in neuroblastomas and glioblastomas; and ALK fusion proteins have occurred in anaplastic large cell lymphoma. The study of ALK fusion proteins has also raised the possibility of new therapeutic treatments for patients with ALK-positive malignancies. (Pulford et al., Cell. Mol. Life Sci. 61:2939-2953 (2004)).

In spite of numerous treatment options for cancer patients, there remains a need for effective and safe therapeutic agents and a need for their preferential use in combination therapy. The compounds 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) are novel compounds that selectively inhibit phosphatidylinositol 3-kinase activity. It has been surprisingly found that these specific PI3K inhibitors have a strong beneficial synergistic interaction and improved anti-proliferative activity when used in combination with anaplastic lymphoma kinase (ALK) inhibitors. It is therefore an object of the present invention to provide for a medicament to improve treatment of cancer.

### SUMMARY OF THE INVENTION

The present invention relates to a pharmaceutical combination comprising: (a) a phosphatidylinositol-3-kinase (PI3K) inhibitor selected from 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and (b) an anaplastic lymphoma kinase (ALK) inhibitor, particularly for separate, simultaneous or sequential use for the treatment or prevention of a proliferative disease.

In one embodiment, the present invention relates to the use of a COMBINATION OF THE INVENTION for the preparation of a pharmaceutical composition or medicament for the treatment or prevention of a proliferative disease.

In one embodiment, the present invention relates to the use of a COMBINATION OF THE INVENTION for the treatment or prevention of a proliferative disease.

In one embodiment, the present invention relates to a method of treating or preventing a proliferative disease in a subject comprising administering to said subject a therapeutically effective amount of a COMBINATION OF THE INVENTION.

In one embodiment, the present invention relates to a pharmaceutical composition or combined preparation, comprising the COMBINATION OF THE INVENTION which is jointly therapeutically effective against a proliferative disease, and optionally at least one pharmaceutically acceptable carrier.

In one embodiment, the present invention relates to a combined preparation comprising (a) one or more dosage units of a PI3K inhibitor selected from COMPOUND A, COMPOUND B, COMPOUND C or any pharmaceutically acceptable salt thereof and (b) one or more dosage units of an ALK inhibitor for use in the treatment or prevention of a proliferative disease.

In one embodiment, the present invention provides a commercial package comprising as active ingredients of COMBINATION OF THE INVENTION, together with instructions for simultaneous, separate or sequential administration of said combination to a patient in need thereof for use in the treatment or prevention of a proliferative disease, particularly a cancer.

In one embodiment, the present invention provides a commercial package comprising as active ingredient a phosphatidylinositol-3-kinase (PI3K) inhibitor selected from 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and instructions for simultaneous, separate or sequential administration of said active ingredient with an anaplastic lymphoma kinase inhibitor to a patient in need thereof for use in the treatment or prevention of a proliferative disease, particularly a cancer.

### DETAILED DESCRIPTION OF THE FIGURES

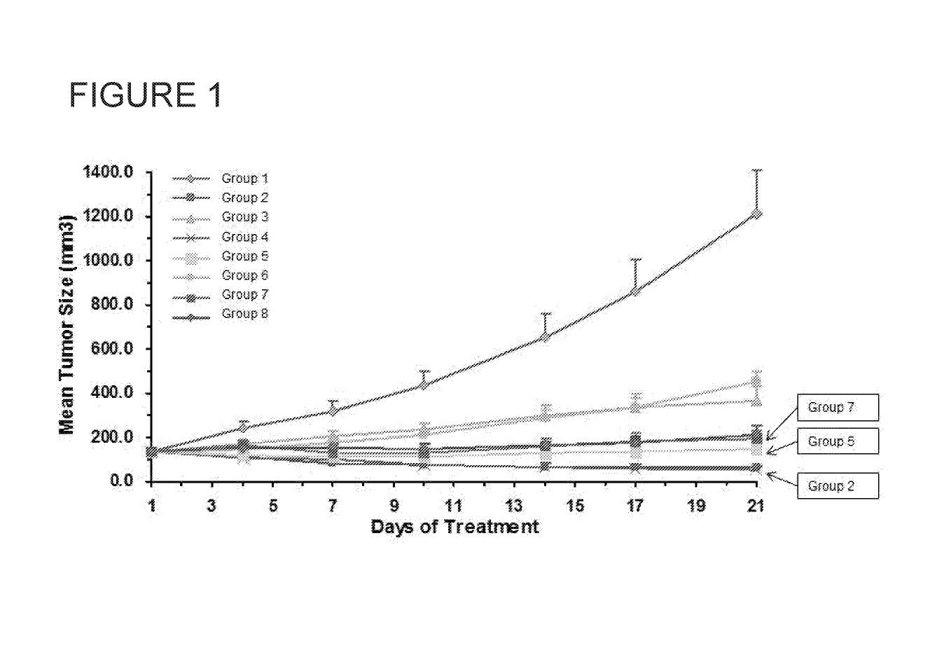

FIG. 1 shows tumor growth curves for female Mus Musculus nu/nu mice inoculated subcutaneously with primary human lung cancer LUF1656 cells during treatment with Vehicle (Group 1), a combination of COMPOUND B monohydrochloride plus COMPOUND D salt (Group 2), COMPOUND B monohydrochloride single agent (Group 3), a combination of COMPOUND B monohydrochloride plus COMPOUND D salt (Group 4), a combination of COMPOUND B monohydrochloride plus crizotinib (Group 5), crizotinib single agent (Group 6), and COMPOUND D salt single agent (Groups 7 and 8) for the experiment described in Example 2.

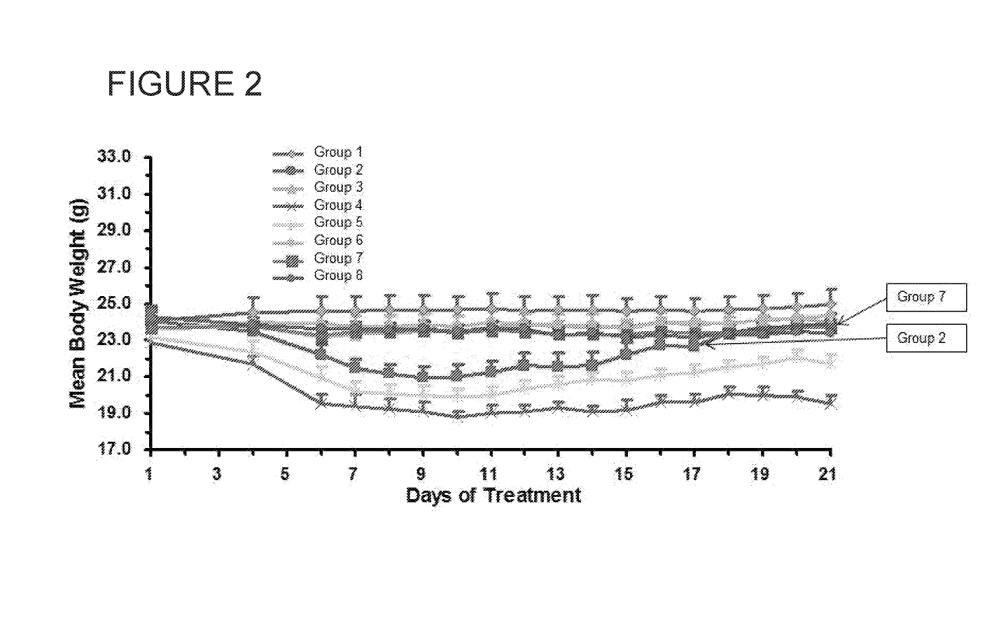

FIG. 2 shows mean body weight change curves for female Mus Musculus nu/nu mice inoculated subcutaneously with primary human lung cancer LUF1656 cells during treatment with Vehicle (Group 1), a combination of COMPOUND B monohydrochloride plus COMPOUND D salt (Group 2), COMPOUND B monohydrochloride single agent (Group 3), a combination of COMPOUND B monohydrochloride plus COMPOUND D salt (Group 4), a combination of COMPOUND B monohydrochloride plus crizotinib (Group 5), crizotinib single agent (Group 6), and COMPOUND D salt single agent (Groups 7 and 8) for the experiment described in Example 2.

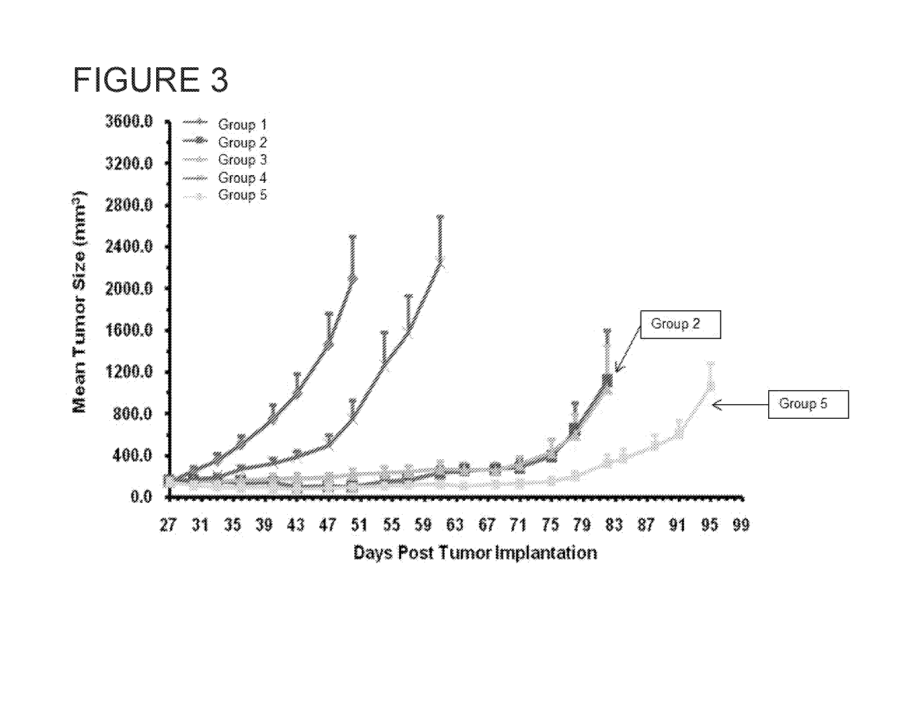

FIG. 3 shows tumor growth curves for female Mus Musculus nu/nu mice inoculated subcutaneously with primary human lung cancer LUF1656 cells during treatment with Vehicle (Group 1), the combination of COMPOUND C plus COMPOUND D salt (Group 2), COMPOUND D salt single agent (Group 3), COMPOUND C single agent (Group 4), and the combination of COMPOUND B monohydrochloride plus COMPOUND D salt (Group 5) for the first phase of the experiment described in Example 3.

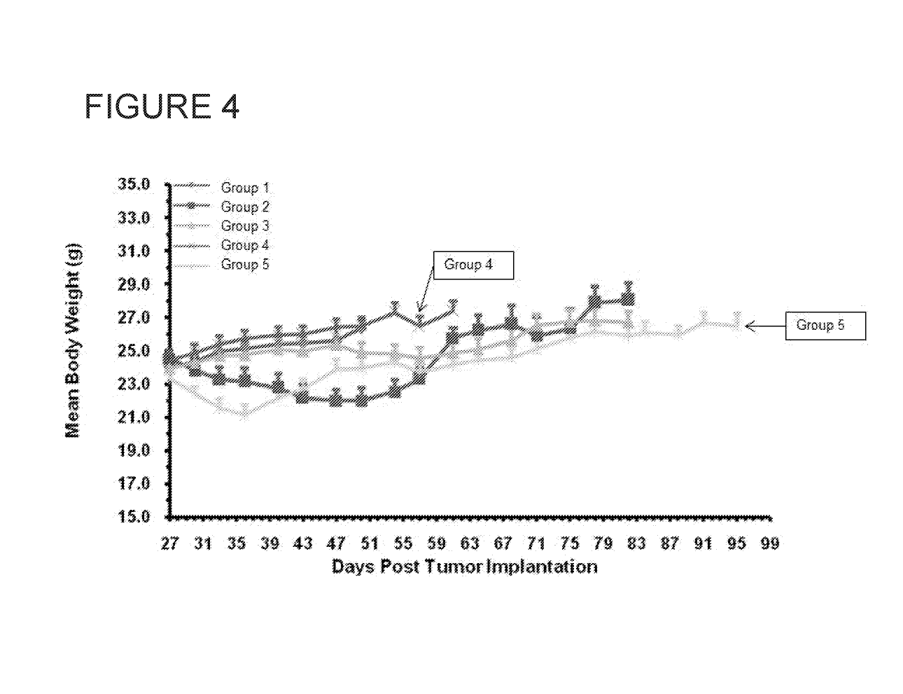

FIG. 4 shows mean body weight change curves for female Mus Musculus nu/nu mice inoculated subcutaneously with primary human lung cancer LUF1656 cells during treatment with Vehicle (Group 1), the combination of COMPOUND C plus COMPOUND D salt (Group 2), COMPOUND D salt single agent (Group 3), COMPOUND C single agent (Group 4), and the combination of COMPOUND B monohydrochloride plus COMPOUND D salt (Group 5) for the first phase of the experiment described in Example 3.

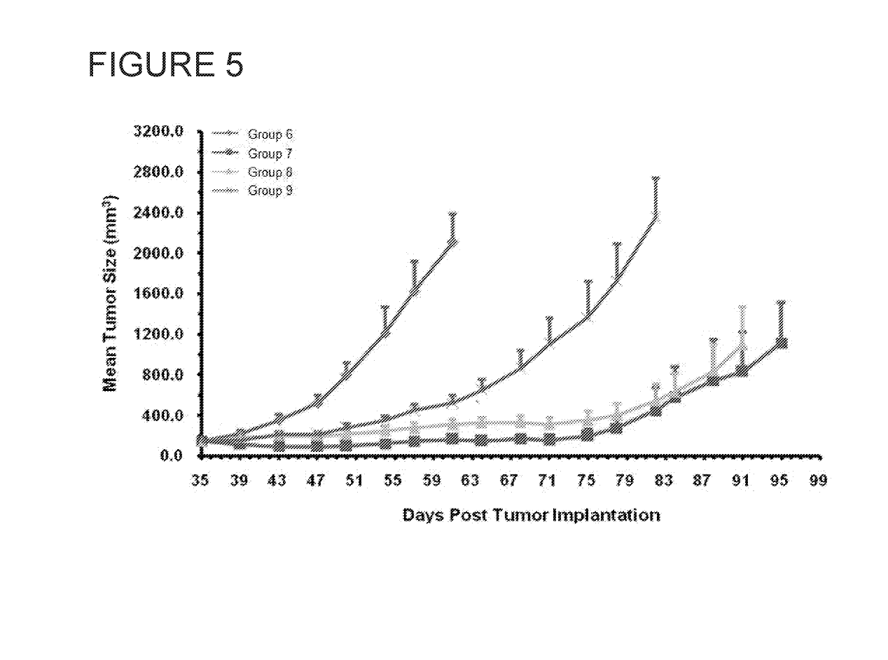

FIG. 5 shows tumor growth curves for female Mus Musculus nu/nu mice inoculated subcutaneously with primary human lung cancer LUF1656 cells during treatment with Vehicle (Group 6), the combination of COMPOUND C plus COMPOUND D salt (Group 7), COMPOUND D salt single agent (Group 8), and crizotinib single agent (Group 9) for the second phase of the experiment described in Example 3.

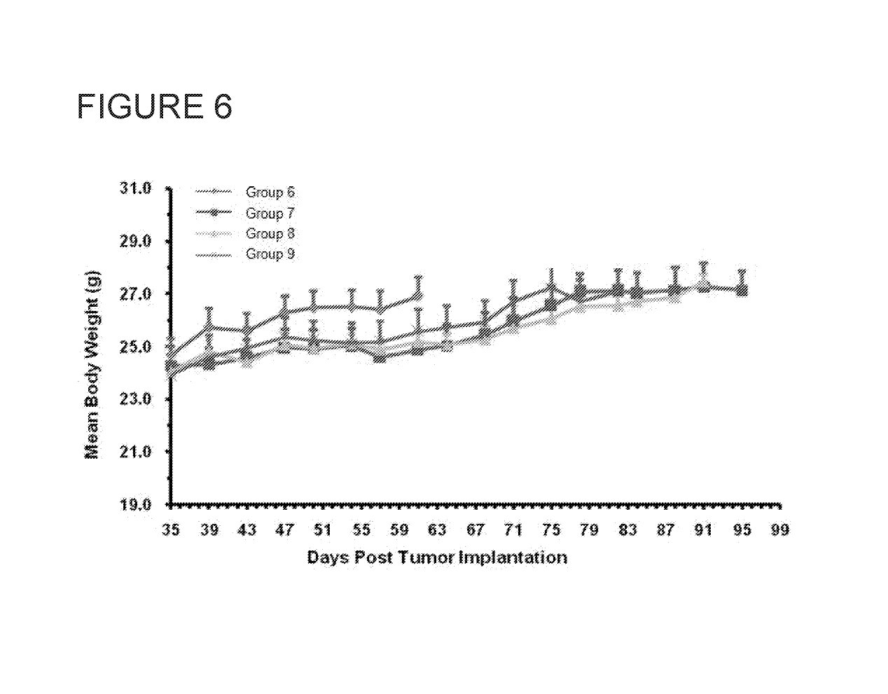

FIG. 6 shows mean body weight change curves for female Mus Musculus nu/nu mice inoculated subcutaneously with primary human lung cancer LUF1656 cells during treatment with Vehicle (Group 6), the combination of COMPOUND C plus COMPOUND D salt (Group 7), COMPOUND D salt single agent (Group 8), and crizotinib single agent (Group 9) for the second phase of the experiment described in Example 3.

### DETAILED DESCRIPTION OF THE INVENTION

The present invention relates to a pharmaceutical combination comprising: (a) a phosphatidylinositol-3-kinase (PI3K) inhibitor selected from 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and (b) an anaplastic lymphoma kinase (ALK) inhibitor, particularly for separate, simultaneous or sequential use for the treatment or prevention of a proliferative disease.

The general terms used herein are defined with the following meanings, unless explicitly stated otherwise:

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “combination” or “pharmaceutical combination” as used herein defines either a fixed combination in one dosage unit form or a kit of parts for the combined administration where the therapeutic agents, e.g. the phosphatidylinositol 3-kinase inhibitor and the anaplastic lymphoma kinase inhibitor, may be administered independently at the same time or separately within time intervals that allow that the therapeutic agents show a cooperative, e.g., synergistic, effect.

The term “combined administration” as used herein is defined to encompass the administration of the selected therapeutic agents to a single patient, and are intended to include treatment regimens in which the therapeutic agents are not necessarily administered by the same route of administration or at the same time.

The term “fixed combination” means that the therapeutic agents, e.g, the phosphatidylinositol 3-kinase inhibitor and the anaplastic lymphoma kinase inhibitor, are administered to a patient simultaneously in the form of a single entity or dosage form.

The term “a combined preparation” is defined herein to refer to especially a “kit of parts” in the sense that the therapeutic agents (a) and (b) as defined above can be dosed independently or by use of different fixed combinations with distinguished amounts of the therapeutic agents (a) and (b), i.e., simultaneously or at different time points. The parts of the kit of parts can then e.g., be administered simultaneously or chronologically staggered, that is at different time points and with equal or different time intervals for any part of the kit of parts. The ratio of the total amounts of the therapeutic agent (a) to the therapeutic agent (b) to be administered in the combined preparation can be varied, e.g., in order to cope with the needs of a patient sub-population to be treated or the needs of the single patient.

The term “pharmaceutically acceptable” is defined herein to refer to those compounds, materials, biologic agents, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

The term “pharmaceutical composition” is defined herein to refer to a mixture or solution containing at least one therapeutic agent to be administered to a subject, e.g., a mammal or human, in order to prevent or treat a particular disease or condition affecting the mammal.

The term “phosphatidylinositol 3-kinase inhibitor” or “PI3K inhibitor” is defined herein to refer to a compound which targets, decreases or inhibits phosphatidylinositol 3-kinase.

The term “anaplastic lymphoma kinase inhibitor” or “ALK inhibitor” is defined herein to refer to a compound or biologic agent which targets, decreases or inhibits the synthesis or biological activity of anaplastic lymphoma kinase (ALK).

The term “treating” or “treatment” as used herein comprises a treatment relieving, reducing or alleviating at least one symptom in a subject or effecting a delay of progression of a proliferative disease. For example, treatment can be the diminishment of one or several symptoms of a proliferative disease or complete eradication of a proliferative disease, such as cancer. Within the meaning of the present invention, the term “treat” also denotes to arrest, delay the onset (i.e., the period prior to clinical manifestation of a proliferative disease) and/or reduce the risk of developing or worsening a proliferative disease. The term “prevention” is used herein to mean prevent, delay or treat, or all, as appropriate, development or continuance or aggravation of a proliferative disease in a subject.

The term “joint therapeutic effect” or “jointly therapeutically effective” means that the therapeutic agents of the combination may be given separately (in a chronologically staggered manner, especially a sequence-specific manner) in such time intervals that they prefer, in the warm-blooded animal, especially human, to be treated, still show a (preferably synergistic) interaction (joint therapeutic effect). Whether this is the case can, inter alia, be determined by following the blood levels, showing that both or all therapeutic agents (compound or antibody) are present in the blood of the human to be treated at least during certain time intervals.

The term “effective amount” or “therapeutically effective amount” of a combination of therapeutic agents is an amount sufficient to provide an observable improvement over the baseline clinically observable signs and symptoms of the proliferative disease treated with the combination.

The term “synergistic effect” as used herein refers to action of two therapeutic agents such as, for example, (a) a phosphatidylinositol 3-kinase (PI3K) inhibitor, and (b) an ALK inhibitor, producing an effect, for example, slowing the symptomatic progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively. Synergy may be further shown by calculating the synergy score of the combination according to methods known by one of ordinary skill.

The term “subject” or “patient” as used herein includes animals, which are capable of suffering from or afflicted with any proliferative disease, particularly a cancer. Examples of subjects include mammals, e.g., humans, dogs, cows, horses, pigs, sheep, goats, cats, mice, rabbits rats and transgenic non-human animals. In the preferred embodiment, the subject is a human, e.g., a human suffering from, at risk of suffering from, or potentially capable of suffering from a proliferative disease (particularly a cancer).

The term “about” or “approximately” shall have the meaning of within 10%, more preferably within 5%, of a given value or range.

A “pharmaceutically acceptable salt”, as used herein, unless otherwise indicated, includes salts of acidic and basic groups which may be present in the compounds of the present invention. Such salts can be prepared in situ during the final isolation and purification of the compounds, or by separately reacting the base or acid functions with a suitable organic or inorganic acid or base, respectively. Suitable salts of the compound include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2 hydroxyethanesulfonate, lactate, maleate, methanesulfonate, nicotinate, 2 naphth-alenesulfonate, oxalate, pamoate, pectinate, persulfate, 3 phenylproionate, picrate, pivalate, propionate, succinate, sulfate, tartrate, thiocyanate, p toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others.

The present invention relates to a pharmaceutical combination comprising: (a) a phosphatidylinositol-3-kinase inhibitor selected from 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and (b) an anaplastic lymphoma kinase inhibitor, particularly for separate, simultaneous or sequential use for the treatment or prevention of a proliferative disease.

Phosphatidylinositol-3-kinase (PI3K) inhibitors suitable for the present invention are selected from 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof.

WO2006/122806 describes imidazoquinoline derivatives, which have been described to inhibit the activity of PI3K. The compound 2-methyl-2-[4-(3-methyl-2-oxo-8-quinolin-3-yl-2,3-dihydro-imidazo[4,5-c]quinolin-1-yl)-phenyl]-propionitrile (hereinafter “COMPOUND A”) has the chemical structure of formula (I)

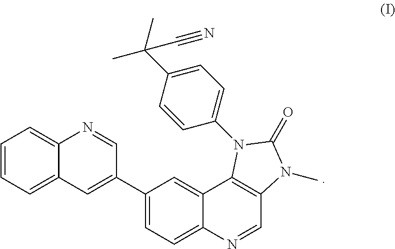 [SMILES: Cn1c(=O)n(-c2ccc(C(C)(C)C#N)cc2)c2c3cc(-c4cnc5ccccc5c4)ccc3ncc21]

The compound, its utility as a PI3K inhibitor and synthesis of 2-methyl-2-[4-(3-methyl-2-oxo-8-quinolin-3-yl-2,3-dihydro-imidazo[4,5-c]quinolin-1-yl)-phenyl]-propionitrile and its monotosylate salt are described in WO2006/122806, which is hereby incorporated by reference in its entirety hereto, for instance in Example 7 and Example 152-3 respectively. COMPOUND A may be present in the form of the free base or any pharmaceutically acceptable salt thereto. Preferably, COMPOUND A is in the form of its monotosylate salt.

WO07/084786 describes specific pyrimidine derivatives which have been found to inhibit the activity of PI3K. The compound 5-(2,6-di-morpholin-4-yl-pyrimidin-4-yl)-4-trifluoromethyl-pyridin-2-ylamine (hereinafter “COMPOUND B”) has the chemical structure of formula (II)

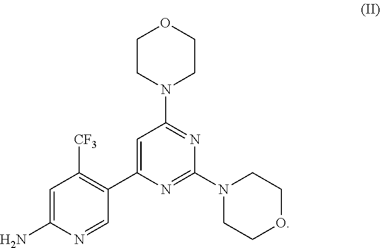 [SMILES: Nc1cc(C(F)(F)F)c(-c2cc(N3CCOCC3)nc(N3CCOCC3)n2)cn1]

The compound, its salts, its utility as a PI3K inhibitor and synthesis of the compound 5-(2,6-di-morpholin-4-yl-pyrimidin-4-yl)-4-trifluoromethyl-pyridin-2-ylamine are described in WO 2007/084786, which is hereby incorporated by reference in its entirety hereto, for instance in Example 10. COMPOUND B may be present in the form of the free base or any pharmaceutically acceptable salt thereto. Preferably, COMPOUND B is in the form of its hydrochloride salt.

WO2010/029082 describes specific 2-carboxamide cycloamino urea derivatives which have been found to be highly selective for the alpha isoform of PI3K. The compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (hereinafter “COMPOUND C”) has the chemical structure of formula (III)

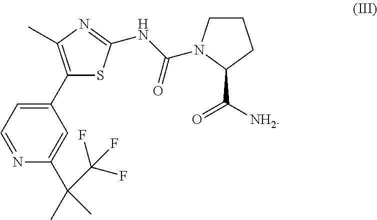 [SMILES: Cc1nc(NC(=O)N2CCC[C@H]2C(N)=O)sc1-c1ccnc(C(C)(C)C(F)(F)F)c1]

The compound, its salts, its utility as an alpha-isoform selective PI3K inhibitor and synthesis of the compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) are described in WO2010/029082, which is hereby incorporated by reference in its entirety, for instance in Example 15. COMPOUND C may be present in the form of the free base or any pharmaceutically acceptable salt thereto. Preferably, COMPOUND C is in the form of its free base.

Anaplastic lymphoma kinase (ALK) inhibitors are known in the art. WO2008/073687 describes novel pyrimidine derivatives which have been found to inhibit the activity of anaplastic lymphoma kinase (ALK). Specific ALK inhibitors suitable for the present invention, their preparation and suitable pharmaceutical formulations containing the same are described in WO2008/073687 and include compounds of formula (IV):

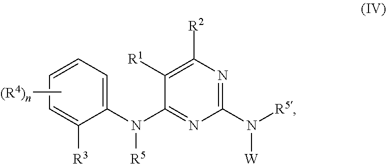

wherein

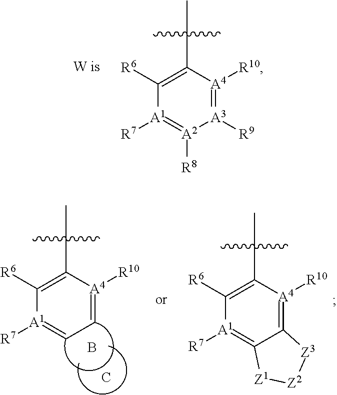

A¹ and A⁴ are independently C or N;
each A² and A³ is C, or one of A² and A³ is N when R⁶ and R⁷ form a ring;
B and C are independently an optionally substituted 5-7 membered carbocyclic ring, aryl, heteroaryl or heterocyclic ring containing N, O or S;
Z¹, Z² and Z³ are independently NR¹¹, C═O, CR—OR, (CR₂)₁₋₂ or ═C—R¹²;
R¹ and R² are independently halo, OR¹², NR(R¹²), SR¹², or an optionally substituted C₁₋₆ alkyl, C₂₋₆alkenyl or C₂₋₆ alkynyl; or one of R¹ and R² is H;
R³ is (CR₂)₀₋₂SO₂R¹², (CR₂)₀₋₂SO₂NRR¹², (CR₂)₀₋₂CO₁₋₂R¹², (CR₂)₀₋₂CONRR¹² or cyano;
R⁴, R⁶, R⁷ and R¹⁰ are independently an optionally substituted C₁₋₆ alkyl, C₂₋₆ alkenyl or C₂₋₆ alkynyl; OR¹², NR(R¹²), halo, nitro, SO₂R¹², (CR₂)ₚR¹³ or X; or R⁴, R⁷ and R¹⁰ are independently H;
R, R⁵ and R^{5′} are independently H or C₁₋₆alkyl;
R⁸ and R⁹ are independently C₁₋₆alkyl, C₂₋₆ alkenyl, C₂₋₆ alkynyl, halo or X, or one of R⁸ and R⁹ is H when R¹ and R² form a ring; and provided one of R⁸ and R⁹ is X;
alternatively, R¹ and R², or R⁶ and R⁷, R⁷ and R⁸, or R⁹ and R¹⁰, when attached to a carbon atom may form an optionally substituted 5-7 membered monocyclic or fused carbocyclic ring, aryl, or heteroaryl or heterocyclic ring comprising N, O and/or S; or R⁷, R⁸, R⁹ and R¹⁰ are absent when attached to N;
R¹¹ is H, C₁₋₆ alkyl, C₂₋₆ alkenyl, (CR₂)ₚCO₁₋₂R, (CR₂)ₚOR, (CR₂)ₚR¹³, (CR₂)ₚNRR¹², (CR₂)ₚCONRR¹² or (CR₂)ₚSO₁₋₂R¹²;
R¹² and R¹³ are independently an optionally substituted 3-7 membered saturated or partially unsaturated carbocyclic ring, or a 5-7 membered heterocyclic ring comprising N, O and/or S; aryl or heteroaryl; or R¹² is H, C₁₋₆ alkyl;
X is (CR₂)_{q}Y, cyano, CO₁₋₂R¹², CONR(R¹²), CONR(CR₂)ₚNR(R¹²), CONR(CR₂)ₚOR¹², CONR(CR₂)ₚSR¹², CONR(CR₂)ₚS(O)₁₋₂R¹² or (CR₂)₁₋₆NR(CR₂)ₚOR¹²;
Y is an optionally substituted 3-12 membered carbocyclic ring, a 5-12 membered aryl, or a 5-12 membered heteroaryl or heterocyclic ring comprising N, O and/or S and attached to A² or A³ or both via a carbon atom of said heteroaryl or heterocyclic ring when q in (CR₂)_{q}Y is 0; and
n, p and q are independently 0-4,
   and pharmaceutically acceptable salts thereof. The radicals and symbols as used in the definition of a compound of formula (IV) have meanings as disclosed in WO2008/073687, which publication is hereby incorporated into the present application by reference in its entirety.

A preferred ALK inhibitor for use in the present invention is a compound which is specifically described in WO2008/073687. A very preferred ALK inhibitor for use in the present invention is the compound 5-Chloro-N2-(2-isopropoxy-5-methyl-4-piperidin-4-yl-phenyl)-N4-[2-(propane-2-sulfonyl)-phenyl]-pyrimidine-2,4-diamine (hereinafter “COMPOUND D”) having the chemical structure of formula (V):

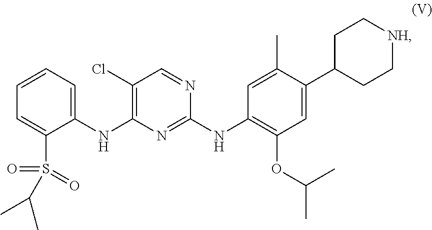 [SMILES: Cc1cc(Nc2ncc(Cl)c(Nc3ccccc3S(=O)(=O)C(C)C)n2)c(OC(C)C)cc1C1CCNCC1]

or a pharmaceutically acceptable salt thereof. COMPOUND D and its utility as an ALK inhibitor are described in WO2008/073687, which is hereby incorporated by reference in its entirety. The synthesis of COMPOUND D is, for instance, described in WO2008/073687 as Example 7, compound 66.

Suitable ALK inhibitors for use in the present invention also include:
(a) Crizotinib (also known as PF02341066 and sold under the tradename XALKORI® by Pfizer) having the chemical structure of formula (VI):

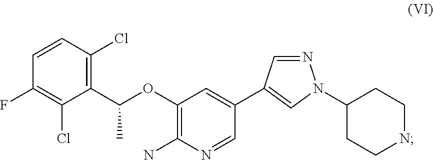

(b) Alectinib (also known as CH5424802) having the chemical structure of formula (VII):

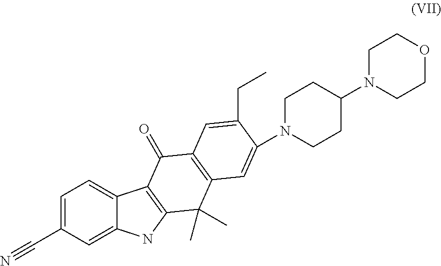 [SMILES: CCc1cc2c(cc1N1CCC(N3CCOCC3)CC1)C(C)(C)c1[nH]c3cc(C#N)ccc3c1C2=O]

The chemical structure and synthesis of Alectinib is described in PCT Application WO WO2010/143664 (Chugai), which is hereby incorporated by reference in its entirety;
(c) 5-Chloro-N4-[2-(isopropylsulfonyl)phenyl]-N2-[2-methoxy-4-[4-(4-methylpiperazin-1-yl)piperidin-1-yl]phenyl]pyrimidine-2,4-diamine (also known as TAE684) having the chemical structure of formula (VIII):

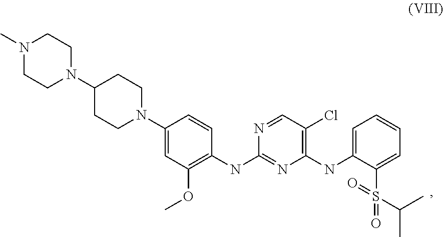 [SMILES: COc1cc(N2CCC(N3CCN(C)CC3)CC2)ccc1Nc1ncc(Cl)c(Nc2ccccc2S(=O)(=O)C(C)C)n1]

The chemical structure and synthesis of TAE684 is described in PCT Application WO WO2005/016894 (Novartis), which is hereby incorporated by reference in its entirety;
(d) CEP28122 having the chemical name (1S,2S,3R,4R)-3-[(5-Chloro-2-[[(7S)-1-methoxy-7-(morpholin-4-yl)-6,7,8,9-tetrahydro-5H-benzocyclohepten-2-yl]amino]pyrimidin-4-yl)amino]bicyclo[2.2.1]hept-5-ene-2-carboxamide mesylate hydrochloride and the chemical structure of formula (IX):

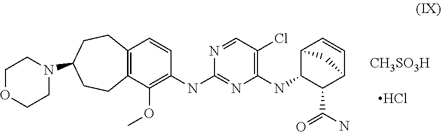

The chemical structure and synthesis of CEP28122 is described in PCT Application WO WO2008/051547 (Cephalon), which is hereby incorporated by reference in its entirety; and
(e) X-396 (by the clinical-stage company Xcovery);
   or any pharmaceutically acceptable salt thereof.

The structure of the active ingredients identified by code numbers, generic or trade names may be taken from the actual edition of the standard compendium “The Merck Index” or from databases, e.g., Patents International (e.g, IMS World Publications). The corresponding content thereof is hereby incorporated by reference.

Hereinafter, the pharmaceutical combination comprising (a) a PI3K inhibitor selected from 2-methyl-2-[4-(3-methyl-2-oxo-8-quinolin-3-yl-2,3-dihydro-imidazo[4,5-c]quinolin-1-yl)-phenyl]-propionitrile (COMPOUND A), 5-(2,6-di-morpholin-4-yl-pyrimidin-4-yl)-4-trifluoromethyl-pyridin-2-ylamine (COMPOUND B), (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (COMPOUND C) or any pharmaceutically acceptable salt thereof, and (b) an ALK inhibitor will be referred to as a COMBINATION OF THE INVENTION.

Unless otherwise specified, or clearly indicated by the text, or not applicable, reference to therapeutic agents useful in the COMBINATION OF THE INVENTION includes both the free base of the compounds, and all pharmaceutically acceptable salts of the compounds.

Unless otherwise specified, or clearly indicated by the text, or not applicable, reference to therapeutic agents useful in the COMBINATION OF THE INVENTION further includes the additional embodiments wherein the PI3K inhibitor is specifically COMPOUND A or any of its pharmaceutically acceptable salts, the embodiment wherein the PI3K inhibitor is specifically COMPOUND B or any of its pharmaceutically acceptable salts, and the embodiment wherein the PI3K inhibitor is specifically COMPOUND C or any of its pharmaceutically acceptable salts.

In one embodiment, the COMBINATION OF THE INVENTION comprises (a) a PI3K inhibitor selected from COMPOUND A, COMPOUND B, COMPOUND C or any pharmaceutically acceptable salt thereof, and (b) an ALK inhibitor selected from a compound of formula (IV), crizotinib, alectinib, 5-Chloro-N4-[2-(isopropylsulfonyl)phenyl]-N2-[2-methoxy-4-[4-(4-methylpiperazin-1-yl)piperidin-1-yl]phenyl]pyrimidine-2,4-diamine, CEP28122, X396 or any pharmaceutically acceptable salt thereof.

In a preferred embodiment, the COMBINATION OF THE INVENTION comprises combination of a PI3K inhibitor selected from COMPOUND A, COMPOUND B, COMPOUND C or a pharmaceutically acceptable salt thereof, and (b) an ALK inhibitor compound of formula (IV), particularly COMPOUND D, or a pharmaceutically acceptable salt thereof.

In a preferred embodiment, the COMBINATION OF THE INVENTION comprises a combination of a PI3K inhibitor selected from COMPOUND A, COMPOUND B, COMPOUND C or a pharmaceutically acceptable salt thereof, and (b) crizotinib or a pharmaceutically acceptable salt thereof.

The present invention particularly pertains to a COMBINATION OF THE INVENTION useful for separate, simultaneous or sequential administration to a subject in need thereof for treating or preventing a proliferative disease. Alternatively stated, the present invention particularly pertains to a COMBINATION OF THE INVENTION for separate, simultaneous or sequential use for treating or preventing a proliferative disease.

The nature of proliferative diseases is multifactorial. Under certain circumstances, drugs with different mechanisms of action may be combined. However, just considering any combination of therapeutic agents having different mode of action does not necessarily lead to combinations with advantageous effects.

It has been surprisingly found that these specific PI3K inhibitors have a strong beneficial synergistic interaction and improved anti-proliferative activity when used in combination with anaplastic lymphoma kinase (ALK) inhibitors, and may be effective for the treatment of a proliferative disease, particularly a cancer. In the present invention, the administration of the COMBINATION OF THE INVENTION is expected to result in a more beneficial treatment, e.g, synergistic or improved anti-proliferative effect, e.g., with regard to the delay of progression of tumor disease or with regard to a change in tumor volume, as compared to either monotherapy.

The COMBINATION OF THE INVENTION is particularly useful for the treatment or prevention of a proliferative disease in a subject in need thereof. The therapeutic agents of the COMBINATION OF THE INVENTION may be separately, simultaneously or sequentially administered to a subject in need thereof. Preferably, these therapeutic agents are administered at therapeutically effective dosages which, when combined, provide a beneficial effect. Thus, in one embodiment of the present invention, the COMBINATION OF THE INVENTION is used for the treatment or prevention of a proliferative disease, particularly a cancer.

In one embodiment, the proliferative disease is cancer. The term “cancer” is used herein to mean a broad spectrum of tumors, including all solid tumors and hematological malignancies. Examples of such tumors include but are not limited to: breast cancer, lung cancer (including small-cell lung cancer and/or non-small cell lung cancer, e.g., squamous cell carcinoma of the lung, large-cell lung carcinoma, lung adenocarcinoma), bronchial cancer, prostate cancer, pancreatic cancer, liver cancer, biliary cancer, cervix cancer, colorectal cancer, hepatocellular cancer, gastric cancer, gastrointestinal cancer, glioma/glioblastoma, endometrial cancer, melanoma, kidney and renal pelvic cancer, urinary bladder cancer, uterine cancer, ovarian cancer, multiple myeloma, brain cancer, head and neck cancer, squamous cell carcinomas, adenocarcinomas, leukemias (including acute myelogenous leukemia, chronic myelogenous leukemia, lymphocytic leukemia, myeloid leukemia), esophageal cancer, hematological and neoplastic diseases (including anaplastic large cell lymphoma, Non-Hodgkin's lymphomas, and diffuse large B-cell lymphoma), inflammatory myofibroblastic tumor, thyroid cancer, neuroblastoma, and combinations thereof.

In a further embodiment, the proliferative disease is breast cancer, lung cancer (including small-cell lung cancer and non-small cell lung cancer, e.g., squamous cell carcinoma of the lung, large-cell lung carcinoma, lung adenocarcinoma), colorectal cancer, esophageal cancer, hematological and neoplastic diseases (including anaplastic large cell lymphoma, Non-Hodgkin's lymphomas, and diffuse large B-cell lymphoma), inflammatory myofibroblastic tumor, thyroid cancer, neuroblastoma or combination thereof.

The COMBINATION OF THE INVENTION inhibits the growth of solid tumors, but also liquid tumors. In a further embodiment of the present invention, the proliferative disease is a solid tumor. The term “solid tumor” especially means melanoma, breast cancer, ovarian cancer, colorectal cancer, and generally gastrointestinal tract, cervix cancer, lung cancer (including small-cell lung cancer and non-small cell lung cancer, e.g., squamous cell carcinoma of the lung, large-cell lung carcinoma, lung adenocarcinoma), head and neck cancer, bladder cancer, and prostate cancer. Further, depending on the tumor type and particular combination used, a decrease of the tumor volume can be obtained. The COMBINATION OF THE INVENTION disclosed herein is also suited to prevent the metastatic spread of tumors and the growth or development of micrometastases.

Further, the COMBINATION OF THE INVENTION disclosed herein is also suitable for the treatment of poor prognosis patients, especially such poor prognosis patients having a cancer which is resistant to treatment employing an ALK inhibitor as a sole therapeutic agent, e.g. a cancer of such patients who initially had responded to treatment with an ALK inhibitor and then relapsed. This cancer may have acquired resistance during prior treatment with one or more ALK inhibitors, e.g., one of those listed above and incorporated herein by reference, e.g, COMPOUND D or its pharmaceutically acceptable salt, or e.g., crizotinib. Thus, in one embodiment, the proliferative disease is a cancer which is resistant to treatment employing an ALK inhibitor as a sole therapeutic agent.

In a further embodiment, the proliferative disease is a cancer selected from breast cancer, lung cancer (including small-cell lung cancer and non-small cell lung cancer, e.g., squamous cell carcinoma of the lung, large-cell lung carcinoma, lung adenocarcinoma), colorectal cancer, esophageal cancer, hematological and neoplastic diseases (including anaplastic large cell lymphoma, Non-Hodgkin's lymphomas, and diffuse large B-cell lymphoma), inflammatory myofibroblastic tumor, thyroid cancer, neuroblastoma, or a combination thereof that is resistant to treatment employing an ALK inhibitor as a sole therapeutic agent. Thus, in a further embodiment, the proliferative disease is lung cancer (including small-cell lung cancer and non-small cell lung cancer, e.g., squamous cell carcinoma of the lung, large-cell lung carcinoma, lung adenocarcinoma) that is resistant to treatment employing an ALK inhibitor as a sole therapeutic agent.

Further, the COMBINATION OF THE INVENTION may be particularly useful for the treatment or prevention of cancers having an ALK fusion gene, amplification or mutations of ALK gene, overexpression or amplification of PI3K alpha, somatic mutation of PIK3CA or germline mutations or somatic mutation of PTEN or mutations and translocation of p85α that serve to up-regulate the p85-p110 complex.

In one embodiment, the present invention relates to the COMBINATION OF THE INVENTION for use in the treatment or prevention of a proliferative disease, particularly a cancer.

In a further embodiment, the present invention is particularly related to COMBINATION OF THE INVENTION for use in the treatment or prevention of a cancer characterized by ALK fusion gene, amplification or mutation of ALK gene, overexpression or amplification of PI3K alpha, somatic mutation of PIK3CA, germline mutations or somatic mutation of PTEN or mutations and translocation of p85α.

In a further embodiment, the present invention relates to the COMBINATION OF THE INVENTION for use in the preparation of a medicament for the treatment or prevention of a proliferative disease, particularly a cancer.

In a further embodiment, the present invention is particularly related to COMBINATION OF THE INVENTION in the preparation of a medicament for the treatment or prevention of a cancer characterized by ALK fusion gene, amplification or mutation of ALK gene, overexpression or amplification of PI3K alpha, somatic mutation of PIK3CA, germline mutations or somatic mutation of PTEN or mutations and translocation of p85α.

In a further embodiment, the present invention relates to the COMBINATION OF THE INVENTION for use in the prevention of the metastatic spread of tumors or the growth or development of micrometastases.

In one embodiment, the present invention relates to a method for treating or preventing a proliferative disease, particularly a cancer, in a subject in need thereof comprising administering to said subject a therapeutically effective amount of a COMBINATION OF THE INVENTION. In each embodiment, COMBINATION OF THE INVENTION is preferably administered in a quantity that is jointly therapeutically effective for the treatment of said proliferative disease in a patient suffering from said proliferative disease.

In a further embodiment, the present invention relates to a method for treating or preventing a proliferative disease, particularly a cancer, in a subject in need thereof comprising comprising simultaneously, separately or sequentially administering to said subject a jointly therapeutically effective amount of a COMBINATION OF THE INVENTION.

In a further embodiment, the present invention is particularly related to a method of treating or preventing a cancer characterized by ALK fusion gene, amplification or mutation of ALK gene, overexpression or amplification of PI3K alpha, somatic mutation of PIK3CA, germline mutations or somatic mutation of PTEN or mutations and translocation of p85α in a subject in need thereof comprising administering to said subject a jointly therapeutically effective amount of a COMBINATION OF THE INVENTION.

In a further embodiment, the present invention relates to a method of treating or preventing a cancer in a subject in need thereof comprising administering to said subject a therapeutically effective amount of a PI3K inhibitor selected from COMPOUND A, COMPOUND B, COMPOUND C or a pharmaceutically acceptable salt thereof and an ALK inhibitor COMPOUND D or a pharmaceutically acceptable salt thereof.

In a further embodiment, the present invention relates to a method of treating or preventing a cancer in a subject in need thereof comprising administering to said subject a therapeutically effective amount of a pharmaceutical combination comprising (a) a PI3K inhibitor selected from COMPOUND A, COMPOUND B, COMPOUND C or a pharmaceutically acceptable salt thereof and (b) an ALK inhibitor crizotinib or a pharmaceutically acceptable salt thereof.

In a further embodiment, the present invention relates to a method for preventing the metastatic spread of tumors or the growth or development of micrometastases in a subject in need thereof comprising comprising simultaneously, separately or sequentially administering to said subject a jointly therapeutically effective amount of a COMBINATION OF THE INVENTION.

In one embodiment, the present invention relates to the use of a COMBINATION OF THE INVENTION for the preparation of a medicament for the treatment or prevention of a proliferative disease, particularly a cancer.

In a further embodiment, the present invention relates to the use of a COMBINATION OF THE INVENTION for the preparation of a medicament for the treatment or prevention of a proliferative disease characterized by ALK fusion gene, amplification or mutation of ALK gene, overexpression or amplification of PI3K alpha, somatic mutation of PIK3CA, germline mutations or somatic mutation of PTEN or mutations and translocation of p85α.

In a further embodiment, the present invention relates to the use of a pharmaceutical combination comprising (a) a PI3K inhibitor selected from COMPOUND A, COMPOUND B, COMPOUND C or a pharmaceutically acceptable salt thereof and (b) an ALK inhibitor COMPOUND D or a pharmaceutically acceptable salt thereof for the preparation of a medicament for the treatment or prevention of a proliferative disease, particularly a cancer.

In a further embodiment, the present invention relates to the use of a pharmaceutical combination comprising (a) a PI3K inhibitor selected from COMPOUND A, COMPOUND B, COMPOUND C or a pharmaceutically acceptable salt thereof and (b) an ALK inhibitor crizotinib or a pharmaceutically acceptable salt thereof for the preparation of a medicament for the treatment or prevention of a proliferative disease, particularly a cancer.

In a further embodiment, the present invention relates to the use for the preparation of a medicament for the prevention of the metastatic spread of tumors or the growth or development of micrometastases.

In one embodiment, the present invention relates to the use of the COMBINATION OF THE INVENTION for the treatment or prevention of a proliferative disease, particularly a cancer.

In a further embodiment, the present invention relates to the use of the COMBINATION OF THE INVENTION for the treatment or prevention of a proliferative disease characterized by ALK fusion gene, amplification or mutation of ALK gene, overexpression or amplification of PI3K alpha, somatic mutation of PIK3CA, germline mutations or somatic mutation of PTEN or mutations and translocation of p85α.

In a further embodiment, the present invention relates to the use of a pharmaceutical combination comprising (a) a PI3K inhibitor selected from COMPOUND A, COMPOUND B, COMPOUND C or a pharmaceutically acceptable salt thereof and (b) an ALK inhibitor COMPOUND D or a pharmaceutically acceptable salt thereof for the treatment or prevention of a proliferative disease, particularly a cancer.

In a further embodiment, the present invention relates to the use of a pharmaceutical combination comprising (a) a PI3K inhibitor selected from COMPOUND A, COMPOUND B, COMPOUND C or a pharmaceutically acceptable salt thereof and (b) an ALK inhibitor crizotinib or a pharmaceutically acceptable salt thereof for the treatment or prevention of a proliferative disease, particularly a cancer.

In a further embodiment, the present invention relates to the use for the preparation of a medicament for the prevention the metastatic spread of tumors or the growth or development of micrometastases.

The administration of a pharmaceutical combination of the invention may result not only in a beneficial effect, e.g. a synergistic therapeutic effect, e.g. with regard to alleviating, delaying progression of or inhibiting the symptoms, but also in further surprising beneficial effects, e.g. fewer side-effects, more durable response, an improved quality of life or a decreased morbidity, compared with a monotherapy applying only one of the pharmaceutically therapeutic agents used in the combination of the invention.

A further benefit is that lower doses of the therapeutic agents of the COMBINATION OF THE INVENTION can be used, for example, that the dosages need not only often be smaller, but are also applied less frequently, or can be used in order to diminish the incidence of side-effects observed with one of the therapeutic agents alone. This is in accordance with the desires and requirements of the patients to be treated.

It can be shown by established test models that a COMBINATION OF THE INVENTION results in the beneficial effects described herein before. The person skilled in the art is fully enabled to select a relevant test model to prove such beneficial effects. The pharmacological activity of a COMBINATION OF THE INVENTION may, for example, be demonstrated in a clinical study or in an animal model as essentially described hereinafter.

It can be shown by established test models that a COMBINATION OF THE INVENTION results in the beneficial effects described herein before. The person skilled in the art is fully enabled to select a relevant test model to prove such beneficial effects. The pharmacological activity of a COMBINATION OF THE INVENTION may, for example, be demonstrated in a clinical study or in an in vivo or in vitro test procedure as essentially described hereinafter.

Suitable clinical studies are in particular, for example, open label, dose escalation studies in patients with a proliferative disease, particularly at cancer. Such studies prove in particular the synergism of the therapeutic agents of the COMBINATION OF THE INVENTION. The beneficial effects on proliferative diseases may be determined directly through the results of these studies which are known as such to a person skilled in the art. Such studies may be, in particular, be suitable to compare the effects of a monotherapy using either therapeutic agent and a COMBINATION OF THE INVENTION. In one embodiment, the dose of the phosphatidylinositol 3-kinase inhibitor selected from COMPOUND A, COMPOUND B, COMPOUND C or a pharmaceutically acceptable salt thereof, is escalated until the Maximum Tolerated Dosage is reached, and the ALK inhibitor is administered with a fixed dose. Alternatively, phosphatidylinositol 3-kinase inhibitor selected from COMPOUND A, COMPOUND B, COMPOUND C or a pharmaceutically acceptable salt thereof, may be administered in a fixed dose and the dose of the ALK inhibitor may be escalated. Each patient may receive doses of the phosphatidylinositol 3-kinase inhibitor either daily or intermittently. The efficacy of the treatment may be determined in such studies, e.g., after 6, 12, 18 or 24 weeks by evaluation of symptom scores every 6 weeks.

Determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients may render impractical the use of this form of testing as a primary model for synergy. However, the observation of synergy in one species can be predictive of the effect in other species and animal models exist, as described herein, to measure a synergistic effect and the results of such studies can also be used to predict effective dose ratio ranges and the absolute doses and plasma concentrations required in other species by the application of pharmacokinetic/pharmacodynamic methods. Established correlations between tumor models and effects seen in man suggest that synergy in animals may be demonstrated, for example, by xenograft models or in appropriate cell lines.

COMPOUND A is generally administered orally at a dose in the range from about 100 mg to 1200 mg, or about 200 mg to 1000 mg, or about 300 mg to 800 mg, or about 400 mg to 600 mg per day in a human adult. The daily dose can be administered on a qd or bid schedule.

COMPOUND B is generally administered orally at a dose in the range from about 30 mg to 300 mg, or about 60 mg to 120 mg, or about 100 mg per day in a human adult. The daily dose can be administered on a qd or bid schedule.

COMPOUND C is generally administered orally at a dose in the range from about from 30 mg to 450 mg per day, for example 100 to 400 mg per day in a human adult. The daily dose can be administered on a qd or bid schedule.

COMPOUND D is generally administered at daily dosages of from about 0.01 to about 100 mg/kg per body weight, or particularly, from about 0.03 to 2.5 mg/kg per body weight. An indicated daily dosage in the larger mammal, e.g. humans, may be in the range from about 0.5 mg to about 2000 mg, or more particularly, from about 0.5 mg to about 100 mg.

Crizotinib may be administered at the suitable dose instructed by the prescribing information when used in the present combinations. However, dose reduction is also a possibility. In the present invention, crizotinib may be administered orally at a dose in the range from about 200 mg to 300 mg B.I.D., or 225 mg to 275 mg B.I.D., and preferably 250 mg B.I.D. in a human adult.

It is understood that each therapeutic agent may be conveniently administered, for example, in one individual dosage unit or divided into multiple dosage units. It is further understood that that each therapeutic agent may be conveniently administered in doses once daily or doses up to four times a day.

In one embodiment, the present invention relates to a pharmaceutical composition or combined preparation comprising a quantity, which is jointly therapeutically effective against a proliferative disease, of the COMBINATION OF THE INVENTION, and optionally at least one pharmaceutically acceptable carrier. In this pharmaceutical composition, the therapeutic agents (i.e, PI3K inhibitor and ALK inhibitor) for use in the combination can be administered in a single formulation or unit dosage form, administered concurrently but separately, or administered sequentially by any suitable route. Preferably, the oral dosage forms of the PI3K inhibitor and the ALK inhibitor are administered concurrently but separately.

A therapeutically effective amount of the therapeutic agents of the COMBINATION OF THE INVENTION may be administered simultaneously or sequentially and in any order, and the components may be administered separately or as a fixed combination. For example, the method of treatment or prevention of a proliferative disease, particularly a cancer, according to the invention may comprise (i) administration of the first therapeutic agent in free or pharmaceutically acceptable salt form and (ii) administration of the second therapeutic agent in free or pharmaceutically acceptable salt form, simultaneously or sequentially in any order, in jointly therapeutically effective amounts, preferably in synergistically effective amounts. The individual therapeutic agents of the COMBINATION OF THE INVENTION can be administered separately at different times during the course of therapy or concurrently in divided or single combination forms. The invention is therefore to be understood as embracing all such regimens of simultaneous or alternating treatment and the term “administering” is to be interpreted accordingly. Preferably, the PI3K inhibitor and the ALK inhibitor are administered separately.

The pharmaceutical composition according to the invention can be prepared in a manner known per se and are those suitable for enteral, such as oral or rectal, and parenteral administration to mammals (warm-blooded animals), including man. Alternatively, when the agents are administered separately, one can be an enteral formulation and the other can be administered parenterally.

Preferably, the pharmaceutical composition comprising the PI3K inhibitor COMPOUND A, COMPOUND B, COMPOUND C or any pharmaceutically acceptable salt thereof is suitable for enteral administration.

The novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, sachets and furthermore ampoules. If not indicated otherwise, these are prepared in a manner known per se, for example by means of conventional mixing, granulating, sugar-coating, dissolving or lyophilizing processes. It will be appreciated that the unit content of one of the therapeutic agents contained in an individual dose of each dosage form need not in itself constitute an effective amount since the necessary effective amount can be reached by administration of a plurality of dosage units.

In preparing the compositions for oral dosage form, any of the usual pharmaceutically acceptable carriers may be employed, such as, for example, water, glycols, oils, alcohols, flavoring agents, preservatives, coloring agents; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed.

One of ordinary skill in the art may select one or more of the aforementioned carriers with respect to the particular desired properties of the dosage form by routine experimentation and without any undue burden. The amount of each carriers used may vary within ranges conventional in the art. The following references which are all hereby incorporated by reference disclose techniques and excipients used to formulate oral dosage forms. See The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2003).

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

The individual therapeutic agents of the COMBINATION OF THE INVENTION may be administered separately at different times during the course of therapy or concurrently in divided or single combination forms. The invention is therefore to be understood as embracing all such regimens of simultaneous or alternating treatment and the term “administering” is to be interpreted accordingly.

The effective dosage of each of the therapeutic agents employed in the COMBINATION OF THE INVENTION may vary depending on the particular compound or pharmaceutical composition employed, the mode of administration, the condition being treated, and the severity of the condition being treated. Thus, the dosage regimen of the COMBINATION OF THE INVENTION is selected in accordance with a variety of factors including the route of administration and the renal and hepatic function of the patient. A clinician or physician of ordinary skill can readily determine and prescribe the effective amount of the single therapeutic agents required to alleviate, counter or arrest the progress of the condition.

The optimum ratios, individual and combined dosages, and concentrations of the therapeutic agents (a) and (b) employed in the COMBINATION OF THE INVENTION that yield efficacy without toxicity are based on the kinetics of the therapeutic agents' availability to target sites, and are determined using methods known to those of skill in the art.

The effective dosage of each of the therapeutic agents employed in the COMBINATION OF THE INVENTION may require more frequent administration of one of the therapeutic agents (s) as compared to the other therapeutic agents (s) in the combination. Therefore, to permit appropriate dosing, packaged pharmaceutical products may contain one or more dosage forms that contain the combination of therapeutic agents, and one or more dosage forms that contain one of therapeutic agents, but not the other therapeutic agent(s) of the combination.

When the therapeutic agents employed in the COMBINATION OF THE INVENTION are applied in the form as marketed as single drugs, their dosage and mode of administration can be in accordance with the information provided on the package insert of the respective marketed drug, if not mentioned herein otherwise.

The optimal dosage of each combination partner for treatment or prevention of a proliferative disease can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art.

The amount of each therapeutic agent of the COMBINATION OF THE INVENTION that may be combined with the carrier materials to produce a single dosage form will vary depending upon the individual treated and the particular mode of administration. In some embodiments the unit dosage forms containing the combination of agents as described herein will contain the amounts of each therapeutic agent of the combination that are typically administered when the therapeutic agents are administered alone.

Frequency of dosage may vary depending on the compound or biologic agent used and the particular condition to be treated or prevented. Patients may generally be monitored for therapeutic effectiveness using assays suitable for the condition being treated or prevented, which will be familiar to those of ordinary skill in the art.

In one embodiment, the present invention relates to a combined preparation comprising (a) one or more dosage units of a PI3K inhibitor selected from COMPOUND A, COMPOUND B, COMPOUND C or any pharmaceutically acceptable salt thereof and (b) one or more dosage units of an ALK inhibitor for use in the treatment or prevention of a proliferative disease.

In one embodiment, the present invention provides a commercial package comprising as active ingredients of COMBINATION OF THE INVENTION and instructions for simultaneous, separate or sequential administration of said combination to a patient in need thereof for use in the treatment or prevention of a proliferative disease, particularly a cancer.

In one embodiment, the present invention provides a commercial package comprising as active ingredient a phosphatidylinositol-3-kinase (PI3K) inhibitor selected from 2-methyl-2-[4-(3-methyl-2-oxo-8-quinolin-3-yl-2,3-dihydro-imidazo[4,5-c]quinolin-1-yl)-phenyl]-propionitrile, 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and instructions for simultaneous, separate or sequential administration of said active ingredient with an anaplastic lymphoma kinase inhibitor to a patient in need thereof for use in the treatment or prevention of a proliferative disease, particularly a cancer.

The following Examples illustrate the invention described above; they are not, however, intended to limit the scope of the invention in any way. The beneficial effects of the pharmaceutical combination of the present invention can also be determined by other test models known as such to the person skilled in the pertinent art.

### Example 1

The effect of combining a phosphatidylinositol-3-kinase inhibitor compound COMPOUND A, COMPOUND B or COMPOUND C with the anaplastic lymphoma kinase inhibitor COMPOUND D on the proliferation of NCI-H2228 non-small cell lung cancer adenocarcinoma cell lines is examined.

The PI3K inhibitors COMPOUND A (monotosylate salt), COMPOUND B (monohydrochloride salt), and COMPOUND C (free base) are obtained in powder form and suspended in a stock of 90% DMSO.

NCI-H2228 cell lines are obtained from American Type Culture Collection (ATCC, Catalog No. CRL-5935).

The tissue-culture treated well plates have a grid layout that includes single agents and the combinations. Cells are plated at 500 cells per well in a 384-well polysterene plate (Corning 3707). Three well plates are prepared in identical format.

The next day (Day 0), the cells are treated with agents in the following groups: (a) a PI3K inhibitor: COMPOUND A (monotosylate salt), COMPOUND B (monohydrochloride salt) or COMPOUND C (free base) or their pharmaceutically acceptable salt alone, (b) an ALK inhibitor: COMPOUND D (phosphate salt) or a pharmaceutically acceptable salt thereof, or (c) a combination of the PI3K inhibitor of (a) and the ALK inhibitor of (b) at a range of concentrations. In the grid layout, cells are treated with the designated agent(s) in a 1:3 dilution series including (a) the IC50 of the drug and (b) three concentrations higher and three concentrations lower than the IC50 of the agent, for a total of 7 concentrations. For example, the dilution series for an agent that has an IC50 of 1 nanomolar is: 27 nM, 9 nM, 3 nM, 1 nM (IC50), 0.333 nM, 0.111 nM, and 0.37 nM of agent.

Each designated agent is added to the cell plate by an acoustic liquid dispense (ATS-100 EDC biosystems) at the ratio of 40 nanoliters of agent into 40 microliters of cells/media.

All well-plates are read using ATPlite reagent read on a Perkin Elmer Envision Plate reader. ATPlite lyses cells and measures ATP activity through luminescence of luciferase that is excited by ATP from cell lysate. The resulting score is correlated to cell number.

On Day 0, one well plate prepared as described above is read as a baseline (“Day 0 read”).

On Day 3, two remaining plates are read and are normalized to the Day 0 read to provide the overall amount of growth inhibition.

Potential synergistic interactions between agent combination are assessed as compared to each single agents and reported as Synergy Score. All synergy calculations are performed using CHALICE software (Zalicus, Cambridge, Mass.).

Following the experimental procedure above, the following Synergy Score results are obtained:

| Combination | Synergy Score | Result |
|---|---|---|
| COMPOUND A + COMPOUND D | 2.02 | Synergistic |
| COMPOUND B + COMPOUND D | 2.34 | Synergistic |
| COMPOUND C + COMPOUND D | 2.96 | Synergistic |

### Example 2

The in-vivo antitumor activity of the phosphatidylinositol-3-kinase inhibitor compound COMPOUND B in combination with either the anaplastic lymphoma kinase inhibitor COMPOUND D or crizotinib is evaluated in the treatment of subcutaneous primary human lung cancer LUF1656 xenograft models in nu/nu mice.

Experiments are performed with female Mus Musculus nu/nu mice (Vital River Laboratories, China) approximately 7-8 weeks of age and 19-24 g body weight at treatment start. All animals are housed in laminar flow rooms at constant temperature (approximately 20-26° C.) and humidity (approximately 40-70%) with 4 or 5 animals in each cage. All animals have free access to irradiation sterilized dry granule food and sterile drinking water. Animals are divided into groups of 10 animals per group.

COMPOUND B in form of its monohydrochloride salt (Novartis) is obtained as a powder and stored at −20° C. 323.3 mg of COMPOUND B monohydrochoride (equivalent to 294 mg of free base) is formulated in 21 ml of 1% Tween 80 and 21 ml 1% methylcellulose (M-0512). COMPOUND B is first sonicated in the 1% Tween 80 and then mixed with the 1% methylcellulose and then stirred for 1 hour and sonicated for 5 minutes to formulate a solution having a COMPOUND B concentration of 7 mg/ml. For this experiment, the COMPOUND B monohydrochloride salt is referred to as “COMPOUND B”. This solution can be stored at 4° C. and protected from light.

COMPOUND D in the form of its salt (Novartis) is obtained as a powder and stored at −20° C. 388 mg of COMPOUND D salt (equivalents to 330 mg of free base) is suspended in 33 ml of 0.5% methylcellulose (M-0512) and 0.5% Tween, is sonicated and then is vortexed to formulate a solution having a COMPOUND D concentration of 10 mg/ml. Additionally, 11 ml of this 10 mg/ml concentration solution is diluted with 11 ml 0.5% methylcellulose and 0.5% Tween 80 and vortexted to form a second solution having a COMPOUND D concentration of 5 mg/ml. For this experiment, the COMPOUND D salt is referred to as “COMPOUND D”. This solution can be stored at room temperature for 1 week and protected from light.

Crizotinib is obtained as a powder and stored at −20° C. 220 mg of crizotinib is suspended in 22 ml 0.5% methylcellulose (MC) and sonicated to form a solution having a crizotinib concentration of 10 mg/ml. This solution can be stored at 4° C. for 1 week and protected from light.

Tumor fragments from stock mice inoculated with LUF1656 primary human lung cancer cells (Crown Bio, China) are harvested and used for inoculation into the nu/nu mice. Each mouse is inoculated subcutaneously at the right flank with one tumor fragment (2-3 mm in diameter) for tumor development. The treatments are started when mean tumor size reach approximately 135 mm³. Animals are routinely monitored and body weight measured twice weekly. Death and observed clinical signs are recorded on the basis of the number of animals within each subset. Animals observed to be in a continuing deteriorating condition or for which the tumor size of individual animal exceeds 1/10 of the body weight are euthanized prior to death or before comatose.

Tumor size is measured twice weekly in two dimensions using a caliper and the volume is expressed in mm³ using the formula: V=0.5 a×b², wherein a and b are the long and short diameters of the tumor respectively. The tumor size is then used for calculation of both T-C and T/C values. T-C is calculated with T as the time (days) required for the mean tumor size of the treatment group to reach a predetermined size (e.g., 400 mm³) and C is the time (in days) for the mean tumor size of the control group to reach the same size. T/C value (in percent is an indication of the anti-tumor effectiveness; T and C are the mean volume of the treated and control groups, respectively, on a given day.

Summary statistics are provided for the tumor volume of each group at each time point. Statistical analysis of difference in tumor volume among the groups is conducted using a one-way ANOVA followed by multiple comparisons using Tukey HSD. Log transformation is performed for homogeneity of variances when necessary. All data is analyzed using SPSS 16.0 P<0.05 is considered to be statistically significant.

The following results are obtained in this experiment:

| | | Tumor Size | | T-C | |
|---|---|---|---|---|---|
| | | (mm³)^{a} at | | (days) | |
| | | Day 21 of | T/C | at 400 | P |
| Group | Treatment | Treatment | (%) | mm³ | value^{b} |
| 1 | Vehicle | 1214.2 ± | — | — | — |
| | | 200.4 | | | |
| 2 | COMPOUND B (35 mg/kg, | 61.9 ± | 5.1 | —^{c} | <0.001 |
| | PO, QD × 21 Days) + | 11.4 | | | |
| | COMPOUND D (25 mg/kg, | | | | |
| | PO, QD × 21 Days) | | | | |
| 3 | COMPOUND B (35 mg/kg, | 365.4 ± | 30.1 | —^{c} | <0.001 |
| | PO, QD × 21 Days) | 62.7 | | | |
| 4 | COMPOUND B (35 mg/kg, | 53.2 ± | 4.4 | —^{c} | <0.001 |
| | PO, QD × 21 Days) + | 10.4 | | | |
| | COMPOUND D (50 mg/kg, | | | | |
| | PO, QD × 21 Days) | | | | |
| 5 | COMPOUND B (35 mg/kg, | 144.9 ± | 11.9 | —^{c} | <0.001 |
| | PO, QD × 21 Days) + | 25.7 | | | |
| | Crizotinib (50 mg/kg, PO, | | | | |
| | QD × 21 Days) | | | | |
| 6 | Crizotinib (50 mg/kg, PO, | 450.8* ± | 37.1 | 7 | 0.010 |
| | QD × 21 Days) | 47.3 | | | |
| 7 | COMPOUND D (25 mg/kg, | 194.9 ± | 16.1 | —^{c} | <0.001 |
| | PO, QD × 21 Days) | 34.6 | | | |
| 8 | COMPOUND D (50 mg/kg, | 213.6 ± | 17.6 | —^{c} | <0.001 |
| | PO, QD × 21 Days) | 38.1 | | | |

Note
^{a}Mean ± SEM;
^{b}vs. vehicle control;
^{c}T-C (days) is not calculated because the mean tumor size till the end of the study is less than 400 mm³;
*the mean tumor size of this group is from 9 animals' data (removed one outlier's data)

FIGS. 1 and 2 show the resulting antitumor activity and body weight changes of these animals in this experiment.

Treatment with 35 mg/kg COMPOUND B (PO, QD×21) plus 25 mg/kg COMPOUND D (PO, QD×21) shows significant anti-tumor activity from Day 4 to Day 21 of treatment compared with vehicle control (p<0.001). The anti-tumor activity of the combination treatment of 35 mg/kg COMPOUND B plus 25 mg/kg COMPOUND D is significantly improved as compared to that of 35 mg/kg COMPOUND B monotherapy from Day 7 to Day 21 of treatment (p<0.001), and also is significantly improved as compared that of 25 mg/kg COMPOUND D monotherapy from Day 10 to Day 21 of treatment (p<0.05, p<0.01 or p<0.001 at different days).

Treatment with 35 mg/kg COMPOUND B (PO, QD×21) plus 50 mg/kg COMPOUND D (PO, QD×21) shows significant anti-tumor activity from Day 4 to Day 21 of treatment compared with vehicle control (p<0.001). The anti-tumor activity of the combination treatment of 35 mg/kg COMPOUND B plus 50 mg/kg COMPOUND D is significantly improved as compared to that of 35 mg/kg COMPOUND B monotherapy from Day 4 to Day 21 of treatment (p<0.05, p<0.01 or p<0.001 at different days), and also significantly improved as compared to that of 50 mg/kg COMPOUND D monotherapy from Day 10 to Day 21 of treatment (p<0.05, p<0.01 or p<0.001 at different days).

In addition, the combination treatments of 35 mg/kg COMPOUND B plus 25 mg/kg COMPOUND D and 35 mg/kg COMPOUND B plus 50 mg/kg COMPOUND D all produced tumor regression in LUF1656 model.

Treatment with 35 mg/kg COMPOUND B (PO, QD×21) plus 50 mg/kg Crizotinib (PO, QD×21) shows significant anti-tumor activity from Day 4 to Day 21 of treatment compared with vehicle control (p<0.01 or p<0.001 at different days). The anti-tumor activity of the combination treatment of 35 mg/kg COMPOUND B plus 50 mg/kg Crizotinib is significantly improved as compared to that of 35 mg/kg COMPOUND B monotherapy from Day 7 to Day 21 of treatment (p<0.05 or p<0.01 at different days), and also significantly improved as compared to that of 50 mg/kg Crizotinib monotherapy from Day 10 to Day 21 of treatment (p<0.05, p<0.01 or p<0.001 at different days).

The body weight of the animals in 35 mg/kg COMPOUND B plus 25 mg/kg COMPOUND D and 35 mg/kg COMPOUND B plus 50 mg/kg COMPOUND D combination treatment groups decreases in the early 2 weeks of treatment and recovers gradually in the later period of treatment. Two animals in 35 mg/kg COMPOUND B plus 25 mg/kg COMPOUND D treated group and all the 10 animals in 35 mg/kg COMPOUND B plus 50 mg/kg COMPOUND D treated group are given one to several dosing holidays during the period of treatment due to severe body weight loss. The body weight of the animals in 35 mg/kg COMPOUND B plus 50 mg/kg Crizotinib combination treatment group also decreases in the early period of treatment, but none of the animals lose over 20% body weight and the body weight of the animals recover gradually in the later period of treatment.

In summary, the combinations of 35 mg/kg COMPOUND B with 25 mg/kg COMPOUND D, 35 mg/kg COMPOUND B with 50 mg/kg COMPOUND D, and 35 mg/kg COMPOUND B in combination with 50 mg/kg Crizotinib all produce significant anti-tumor activity against the primary human lung cancer LUF1656 xenograft model. All the three combination therapies demonstrate significantly better anti-tumor activity than corresponding monotherapy against the primary human lung cancer LUF1656 xenograft model in this study.

### Example 3

The in-vivo antitumor activity (b) of the phosphatidylinositol-3-kinase inhibitor compound COMPOUND B in combination with either the anaplastic lymphoma kinase inhibitor COMPOUND D or crizotinib and (b) of the phosphatidylinositol-3-kinase inhibitor compound COMPOUND C in combination with either the anaplastic lymphoma kinase inhibitor COMPOUND D or crizotinib is evaluated in the treatment of subcutaneous primary human lung cancer LU1656 xenograft models in nu/nu mice.

Experiments are performed with female Mus Musculus nu/nu mice (Vital River Laboratories, China) approximately 7-8 weeks of age and 19-24 g body weight at treatment start. All animals are housed in laminar flow rooms at constant temperature (approximately 20-26° C.) and humidity (approximately 40-70%) with 4 or 5 animals in each cage. All animals have free access to irradiation sterilized dry granule food and sterile drinking water.

Animals are divided into 9 groups. The study is conducted in a first phase and second phase due to the variance in tumor growth rates. In the first part of the study, 5 groups of 10 animals per group and each animal having faster growth tumors. In the second part of the study, 4 groups of 8 animals per group and each animal having slower growth tumors that reach the optimal size for the experiment.

In the first phase of the study:

COMPOUND B in form of its monohydrochloride salt (Novartis) is obtained as a powder and stored at −20° C. 99.1 mg of COMPOUND B monohydrochoride (equivalent to 91 mg of free base) is formulated in 6.5 ml of 1% Tween 80 and 6.5 ml 1% methylcellulose (M-0512). COMPOUND B is first sonicated in the 1% Tween 80 and then mixed with the 1% methylcellulose and then stirred for 1 hour and sonicated for 5 minutes to formulate a solution having a COMPOUND B concentration of 7 mg/ml. For this experiment, the COMPOUND B monohydrochloride salt is referred to as “COMPOUND B”. This solution can be stored at 4° C. and protected from light.

COMPOUND C is obtained as a powder and stored at 4° C. A vehicle is formulated by dissolving 2 g of CMC sodium salt powder low density in 198 ml of sterile water, mixing using a magnetic beater and heating (about 50° C.), adding 1 ml Tween 80, adjusting the pH to 7.6 using NaOh/HCl, and completing the volume to 200 ml. 84 mg of COMPOUND C is suspended in 3.5 ml of this vehicle and stirred/vortexed until homogenous; another 3.5 ml of this vehicle is added and homogenized; and another 7 ml of this vehicle is added and homogenized. To reduce particle size, this solution is sonicated for 1 hour using a sonication probe while cooling in an ice bath. The solution is having a COMPOUND C concentration of 6 mg/ml. This solution can be stored at room temperature for 1 week and protected from light. This solution can be stored at 4° C. for 4 days and protected from light.

COMPOUND D in the form of its salt (Novartis) is obtained as a powder and stored at −20° C. 388 mg of COMPOUND D salt (equivalents to 330 mg of free base) is suspended in 33 ml of 0.5% methylcellulose (M-0512) and 0.5% Tween, is sonicated and then is vortexed to formulate a solution having a COMPOUND D concentration of 10 mg/ml. Additionally, 11 ml of this 10 mg/ml concentration solution is diluted with 11 ml 0.5% methylcellulose and 0.5% Tween 80 and vortexted to form a second solution having a COMPOUND D concentration of 5 mg/ml. For this experiment, the COMPOUND D salt is referred to as “COMPOUND D”. This solution can be stored at room temperature for 1 week and protected from light.

In the second phase of the study:

COMPOUND C is obtained as a powder and stored at 4° C. A vehicle is formulated by dissolving 2 g of CMC sodium salt powder low density in 198 ml of sterile water, mixing using a magnetic beater and heating (about 50° C.), adding 1 ml Tween 80, adjusting the pH to 7.6 using NaOh/HCl, and completing the volume to 200 ml. 40.8 mg of COMPOUND C is suspended in 1.7 ml of this vehicle and stirred/vortexed until homogenous; another 1.7 ml of this vehicle is added and homogenized; and another 3.4 ml of this vehicle is added and homogenized. To reduce particle size, this solution is sonicated for 1 hour using a sonication probe while cooling in an ice bath. The solution is having a COMPOUND C concentration of 6 mg/ml. This solution can be stored at 4° C. for 4 days and protected from light.

COMPOUND D in the form of its salt (Novartis) is obtained as a powder and stored at −20° C. 105.8 mg of COMPOUND D salt (equivalents to 90 mg of free base) is suspended in 18 ml of 0.5% methylcellulose (M-0512) and 0.5% Tween, is sonicated and then is vortexed to formulate a solution having a COMPOUND D concentration of 5 mg/ml. For this experiment, the COMPOUND D salt is referred to as “COMPOUND D”. This solution can be stored at room temperature for 1 week and protected from light.

Crizotinib is obtained as a powder and stored at −20° C. 100 mg of crizotinib is suspended in 10 ml 0.5% methylcellulose (MC) and sonicated to form a solution having a crizotinib concentration of 10 mg/ml. This solution can be stored at 4° C. for 1 week and protected from light.

Tumor fragments from stock mice inoculated with LU1656 primary human lung cancer cells (Crown Bio, China) are harvested and used for inoculation into the nu/nu mice. Each mouse is inoculated subcutaneously at the right flank with one tumor fragment (2-3 mm in diameter) for tumor development. The treatments are started when mean tumor size reach approximately 135 mm³. Animals are routinely monitored and body weight measured twice weekly. Death and observed clinical signs are recorded on the basis of the number of animals within each subset. Animals observed to be in a continuing deteriorating condition or for which the tumor size of individual animal exceeds 3000 mm³ (or for which the mean tumor size of the group exceeds 2000 mm³) are euthanized prior to death or before comatose.

Tumor size is measured twice weekly in two dimensions using a caliper and the volume is expressed in mm³ using the formula: V=0.5 a×b², wherein a and b are the long and short diameters of the tumor respectively. The tumor size is then used for calculation of both T-C and T/C values. T-C is calculated with T as the time (days) required for the mean tumor size of the treatment group to reach a predetermined size (e.g, 500 mm³) and C is the time (in days) for the mean tumor size of the control group to reach the same size. T/C value (in percent is an indication of the anti-tumor effectiveness; T and C are the mean volume of the treated and control groups, respectively, on a given day.

For the first phase, statistical analysis of difference in tumor volume among vehicle group, COMPOUND D alone group, COMPOUND C alone group, the combination of COMPOUND C plus COMPOUND D group and combination of COMPOUND B plus COMPOUND D group are evaluated using a one-way ANOVA test followed by multiple comparisons using Tukey HSD. Log transformation is performed for homogeneity of variances when necessary.

For the second phase, statistical analysis of difference in tumor volume among the groups is evaluated using the same method as the first phase.

The following results are obtained in the first phase of this experiment:

| | | Tumor Size | | T-C | |
|---|---|---|---|---|---|
| | | (mm³)^{a} at | | (days) | |
| | | Day 50 Post | | at | |
| | | Tumor Im- | T/C | 500 | P |
| Group | Treatment | plantation | (%) | mm³ | value^{b} |
| 1 | Vehicle (p.o.; QD × 24) | 2092.2 ± | — | — | — |
| | | 403.2 | | | |
| 2 | COMPOUND C (30 mg/ | 98.2 ± | 4.7 | 40 | <0.001 |
| | kg; p.o.; QD × 29) + | 20.5 | | | |
| | COMPOUND D (50 mg/ | | | | |
| | kg; p.o.; QD × 29) | | | | |
| 3 | COMPOUND D (50 mg/ | 218.2 ± | 10.4 | 40 | <0.001 |
| | kg; p.o.; QD × 40) | 51.3 | | | |
| 4 | COMPOUND C (30 mg/ | 752.5 ± | 36.0 | 11 | 0.064 |
| | kg; p.o.; QD × 35) | 176.3 | | | |
| 5 | COMPOUND B (35 mg/ | 95.6 ± | 4.6 | 52 | <0.001 |
| | kg; p.o.; QD × 40) + | 17.9 | | | |
| | COMPOUND D (25 mg/ | | | | |
| | kg; p.o.; QD × 40) | | | | |

Note
^{a}Mean ± SEM;
^{b}vs. vehicle control (group 1)

The following results are obtained in the second phase of this experiment:

| | | Tumor Size | | | |
|---|---|---|---|---|---|
| | | (mm³)^{a} at | | T-C | |
| | | Day 61 Post | | (days) | |
| | | Tumor Im- | T/C | at 500 | P |
| Group | Treatment | plantation | (%) | mm³ | value^{b} |
| 6 | Vehicle (p.o.; QD × 27) | 2108.3 ± | — | — | — |
| | | 276.8 | | | |
| 7 | COMPOUND C (30 mg/ | 163.2 ± | 7.7 | 36 | <0.001 |
| | kg; p.o.; QD × 40) + | 29.1 | | | |
| | COMPOUND D (25 mg/ | | | | |
| | kg; p.o.; QD × 40) | | | | |
| 8 | COMPOUND D (25 mg/ | 307.4 ± | 14.6 | 34 | <0.001 |
| | kg; p.o.; QD × 40) | 53.2 | | | |
| 9 | Crizotinib (50 mg/kg; | 520.4 ± | 24.7 | 14 | <0.001 |
| | p.o.; QD × 40) | 75.6 | | | |

Note
^{a}Mean ± SEM;
^{b}vs. vehicle control (group 6)

FIGS. 3 and 4 show the resulting antitumor activity and body weight changes of these animals in the first phase of this experiment. FIGS. 5 and 6 show the resulting antitumor activity and body weight changes of these animals in the second phase of this experiment.

In the first phase of the study, treatment with COMPOUND B (35 mg/kg, PO, QD×40) plus COMPOUND D (25 mg/kg, PO, QD×40) shows significant antitumor activity from Day 30 to Day 50 after tumor implantation compared with vehicle control (p<0.01 or p<0.001 at different days). The antitumor activity of the combination treatment (30 mg/kg COMPOUND C in combination with 50 mg/kg COMPOUND D) is significantly improved as compared to that of COMPOUND C (30 mg/kg) monotherapy from Day 36 to Day 61 after tumor implantation (p<0.05, p<0.01 or p<0.001 at different days), but has no statistical difference as compared with COMPOUND D (50 mg/kg) monotherapy.

In the second phase of the study, treatment with COMPOUND C (30 mg/kg, PO, QD×40) plus COMPOUND D (25 mg/kg, PO, QD×40) shows significant antitumor activity from Day 39 to Day 61 after tumor implantation compared with vehicle control (p<0.01 or p<0.001 at different days). Treatment with Crizotinib (50 mg/kg, PO, QD×40) shows significant antitumor activity from Day 47 to Day 61 after tumor implantation compared with vehicle control (p<0.01 or p<0.001 at different days). The antitumor activity of the combination treatment (30 mg/kg COMPOUND C in combination with 25 mg/kg COMPOUND D) is significantly improved as compared to that of COMPOUND D (25 mg/kg) monotherapy at Day 43 and Day 47 after tumor implantation (p<0.05). Also, the antitumor activity of both the combination treatment (30 mg/kg COMPOUND C plus 25 mg/kg COMPOUND D) and COMPOUND D (25 mg/kg) monotherapy is significantly improved as compared to that of Crizotinib (50 mg/kg) monotherapy from Day 43 to Day 82 (p<0.01 or p<0.001 at different days) and from Day 64 to Day 82 (p<0.05 or p<0.01 at different days), respectively.

The duration of treatment for Group 2 (30 mg/kg COMPOUND C plus 50 mg/kg COMPOUND D) is 29 days, less than 40 days as designed due to toxicity. Some animals in Group 3, 5, 7 and 8 are given dosing holidays during the dosing period due to toxicity.

In summary, COMPOUND D at 25 and 50 mg/kg as a single agent, COMPOUND C at 30 mg/kg as a single agent, COMPOUND C (30 mg/kg) in combination with COMPOUND D (25 mg/kg), COMPOUND C (30 mg/kg) in combination with COMPOUND D (50 mg/kg), COMPOUND B (35 mg/kg) in combination with COMPOUND D (25 mg/kg) and Crizotinib at 50 mg/kg as a single agent all produce significant antitumor activity against the primary human lung cancer LU1656 xenograft model. The combination therapy of COMPOUND C (30 mg/kg) plus COMPOUND D (50 mg/kg) demonstrates significantly improved antitumor activity as compared to COMPOUND C (30 mg/kg) monotherapy, but not significantly improved as compared to COMPOUND D (50 mg/kg) monotherapy. Both the combination therapy of COMPOUND C (30 mg/kg) plus COMPOUND D (25 mg/kg) and COMPOUND D (25 mg/kg) monotherapy demonstrate significantly improved antitumor activity as compared to Crizotinib (50 mg/kg) monotherapy against the primary human lung cancer LU1656 xenograft model in this study.

1. A pharmaceutical combination comprising: (a) a phosphatidylinositol-3-kinase (PI3K) inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and (b) an anaplastic lymphoma kinase (ALK) inhibitor.

2. The pharmaceutical combination according to claim 1, wherein the anaplastic lymphoma kinase inhibitor is selected from a compound having Formula (IV) 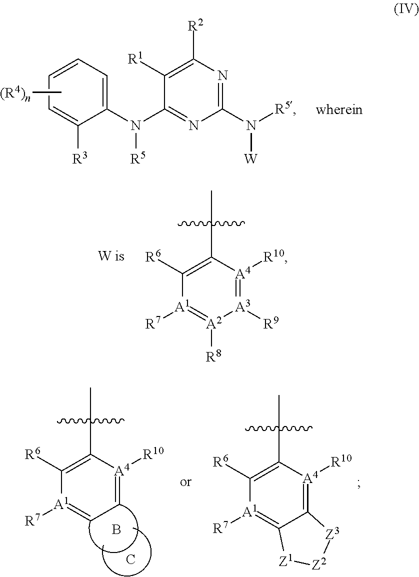
A¹ and A⁴ are independently C or N;
each A² and A³ is C, or one of A² and A³ is N when R⁶ and R⁷ form a ring;
B and C are independently an optionally substituted 5-7 membered carbocyclic ring, aryl, heteroaryl or heterocyclic ring containing N, O or S;
Z¹, Z² and Z³ are independently NR¹¹, C═O, CR—OR, (CR₂)₁₋₂ or ═C—R¹²;
R¹ and R² are independently halo, OR¹², NR(R¹²), SR¹², or an optionally substituted C₁₋₆ alkyl, C₂₋₆ alkenyl or C₂₋₆ alkynyl; or one of R¹ and R² is H;
R³ is (CR₂)₀₋₂SO₂R¹², (CR₂)₀₋₂SO₂NRR¹², (CR₂)₀₋₂CO₁₋₂R¹², (CR₂)₀₋₂CONRR¹² or cyano;
R⁴, R⁶, R⁷ and R¹⁰ are independently an optionally substituted C₁₋₆ alkyl, C₂₋₆ alkenyl or C₂₋₆ alkynyl; OR¹², NR(R¹²), halo, nitro, SO₂R¹², (CR₂)ₚR¹³ or X; or R⁴, R⁷ and R¹⁰ are independently H;
R, R⁵ and R^{5′} are independently H or C₁₋₆alkyl;
R⁸ and R⁹ are independently C₁₋₆ alkyl, C₂₋₆ alkenyl, C₂₋₆ alkynyl, halo or X, or one of R⁸ and R⁹ is H when R¹ and R² form a ring; and provided one of R⁸ and R⁹ is X;
alternatively, R¹ and R², or R⁶ and R⁷, R⁷ and R⁸, or R⁹ and R¹⁰, when attached to a carbon atom may form an optionally substituted 5-7 membered monocyclic or fused carbocyclic ring, aryl, or heteroaryl or heterocyclic ring comprising N, O and/or S; or R⁷, R⁸, R⁹ and R¹⁰ are absent when attached to N;
R¹¹ is H, C₁₋₆ alkyl, C₂₋₆ alkenyl, (CR₂)ₚCO₁₋₂R, (CR₂)ₚOR, (CR₂)ₚR¹³, (CR₂)ₚNRR¹², (CR₂)ₚCONRR¹² or (CR₂)ₚSO₁₋₂R¹²;
R¹² and R¹³ are independently an optionally substituted 3-7 membered saturated or partially unsaturated carbocyclic ring, or a 5-7 membered heterocyclic ring comprising N, O and/or S; aryl or heteroaryl; or R¹² is H, C₁₋₆ alkyl;
X is (CR₂)_{q}Y, cyano, CO₁₋₂R¹², CONR(R¹²), CONR(CR₂)ₚNR(R¹²), CONR(CR₂)ₚOR¹², CONR(CR₂)ₚSR¹², CONR(CR₂)ₚS(O)₁₋₂R¹² or (CR₂)₁₋₆NR(CR₂)ₚOR¹²;
Y is an optionally substituted 3-12 membered carbocyclic ring, a 5-12 membered aryl, or a 5-12 membered heteroaryl or heterocyclic ring comprising N, O and/or S and attached to A² or A³ or both via a carbon atom of said heteroaryl or heterocyclic ring when q in (CR₂)_{q}Y is 0; and
n, p and q are independently 0-4,
crizotinib, alectinib, 5-Chloro-N4-[2-(isopropylsulfonyl)phenyl]-N2-[2-methoxy-4-[4-(4-methylpiperazin-1-yl)piperidin-1-yl]phenyl]pyrimidine-2,4-diamine, CEP28122, X396 or any pharmaceutically acceptable salt thereof.

3. The pharmaceutical combination according to claim 2, wherein the anaplastic lymphoma kinase inhibitor is a compound of formula (IV) that is the compound 5-Chloro-N2-(2-isopropoxy-5-methyl-4-piperidin-4-yl-phenyl)-N4-[2-(propane-2-sulfonyl)-phenyl]-pyrimidine-2,4-diamine or a pharmaceutically acceptable salt thereof.

4. The pharmaceutical combination according to claim 1, for simultaneous, separate or sequential use.

5. (canceled)

6. (canceled)

7. (canceled)

8. A pharmaceutical composition comprising the pharmaceutical combination according to claim 1.

9. A combined preparation comprising: (a) one or more dosage units of a phosphatidylinositol-3-kinase inhibitor selected from, 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof, and (b) one or more dosage units of an anaplastic lymphoma kinase (ALK) inhibitor, for use in the treatment or prevention of a proliferative disease.

10. (canceled)

11. (canceled)

12. A method for treating or preventing a proliferative disease in a subject in need thereof comprising administering to said subject a therapeutically effective amount of (a) a phosphatidylinositol-3-kinase inhibitor selected from 5-(2,6-di-morpholin-4-yl-pyrimidin-4-yl)-4-trifluoromethyl-pyridin-2-ylamine, (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or any pharmaceutically acceptable salt thereof and (b) an anaplastic lymphoma kinase inhibitor.

13. The method according to claim 12, wherein the ALK inhibitor is selected from a compound of formula (IV), crizotinib, alectinib, 5-Chloro-N4-[2-(isopropylsulfonyl)phenyl]-N2-[2-methoxy-4-[4-(4-methylpiperazin-1-yl)piperidin-1-yl]phenyl]pyrimidine-2,4-diamine, CEP28122, X396 or any pharmaceutically acceptable salt thereof.

14. The method according to claim 12, wherein the ALK inhibitor is 5-Chloro-N2-(2-isopropoxy-5-methyl-4-piperidin-4-yl-phenyl)-N4-[2-(propane-2-sulfonyl)-phenyl]-pyrimidine-2,4-diamine or a pharmaceutically acceptable salt thereof.

15. A commercial package comprising as active ingredient a phosphatidylinositol-3-kinase (PI3K) inhibitor according to claim 1 and instructions for simultaneous, separate or sequential administration of said active ingredient with an anaplastic lymphoma kinase inhibitor to a patient in need thereof for use in the treatment or prevention of a proliferative disease.

16. The method according to claim 12, wherein the proliferative disease is a cancer selected from breast cancer, lung cancer (including small-cell lung cancer and non-small cell lung cancer), colorectal cancer, esophageal cancer, hematological and neoplastic diseases (including anaplastic large cell lymphoma, Non-Hodgkin's lymphomas, and diffuse large B-cell lymphoma), inflammatory myofibroblastic tumor, thyroid cancer, neuroblastoma or a combination thereof.

17. The method according to claim 12, wherein the proliferative disease is resistant to treatment with an anaplastic lymphoma kinase inhibitor.

18. The method according to claim 12, wherein the proliferative disease is a cancer having an ALK fusion gene, amplification or mutations of ALK gene, overexpression or amplification of PI3K alpha, somatic mutation of PIK3CA or germline mutations or somatic mutation of PTEN or mutations and translocation of p85α.
